(19) United States
(12) Patent Application Publication
(10) Pub. No.: US 2011/0027873 A1
(43) Pub. Date: Feb. 3, 2011

(54) MICRO-NANO FLUIDIC BIOCHIP FOR ASSAYING BIOLOGICAL SAMPLE

(75) Inventors: Sin Kil Cho, Seoul (KR); Seok Chung, Cambridge, MA (US)

Correspondence Address: SUGHRUE MION, PLLC, 2100 PENNSYLVANIA AVENUE, N.W., SUITE 800, WASHINGTON, DC, 20037, US

(73) Assignee: INCYTO CO., LTD., Cheonan-si, Chungcheongnam-do (KR)

(21) Appl. No.: 12/936,861

(22) PCT Filed: Apr. 10, 2009
(86) PCT No.: PCT/KR09/01854

§ 371 Date: Oct. 7, 2010

(30) Foreign Application Priority Data
Apr. 11, 2008 (KR) 10-2008-0033834

(51) Int. Cl. C12M 1/34; G01N 30/00

(52) U.S. Cl. 435/287.1; 422/69

(57) ABSTRACT

Disclosed is a micro-nano fluidic biochip for assaying a biological sample comprising a first substrate, a second substrate and a third substrate which are sequentially stacked from bottom to top, wherein an upper channel assembly disposed on the second substrate is coupled with the lower channel assembly provided on the first substrate, to form a microfluidic channel, and the microfluidic channel has nano interstices formed at both sides thereof, the nano interstices having a height less than that of the center of the channel.

### FIELD OF THE INVENTION

The present invention relates to a micro-nano fluidic biochip for assaying a biological sample.

### BACKGROUND OF THE INVENTION

A micro-nano fluidic biochip is used for disease diagnosis and biological assays through the procedure of inducing a reaction of a biological sample to be assayed with a test reagent in a nano-scale membrane or channel disposed on a microchip.

U.S. Pat. Nos. 6,242,862, 6,818,455, 6,951,631, 7,153,651 and 7,238,537 (hereinafter, referred to as the '862 patent) disclose various biochips for assaying biological samples. These patents disclose the use of a membrane for sample analysis having good absorptive capacity and containing a reagent efficiently immobilized thereon to enable high signal detection. Specifically, a specific reactive reagent is applied on a membrane, a biological sample is allowed to flow thereto, and the degree of the reaction therebetween is detected. These methods are mainly used to detect a specific component in a qualitative manner, and thus, the quantification of the signal is difficult due to the uneven absorption of the agent and also to the interference by membrane residues after the reaction. Also, a complicated washing procedure is often required.

To solve such problems, U.S. Pat. Nos. 5,885,527, 6,019,944, 6,143,576, 6,156,270, 6,271,040, 6,391,265, 6,767,510 and 6,905,882 (hereinafter, referred to as the '527 patent) disclose the use of a microfluidic channel and a reagent-containing pad in lieu of the membrane, and the fluid flow through the channel is controlled by adjusting the shape of the channel. This method is advantageous because uniform reagent absorption and signal quantification are achievable, but the absorptive capacity of the channel is low, which limits the selection of the pad-type. Also, as the use of a relatively large-size and long channel are required, the amount of the sample that must be used becomes undesirably large (e.g., to about 300 μl or more). Further, because the channel is long and has a complicated shape, a particular means must be used to prevent fluid leakage. In case such a channel is employed, the procedures for the immobilization of a reagent and the use of a color reagent used in conventional systems cannot be employed, and thus, they must be individually developed. Moreover, as the channel has low absorptive capacity, some samples, e.g., urine and saliva, cannot be used, besides the problem that the fabrication of a channel having a complex shape becomes difficult.

### SUMMARY OF THE INVENTION

Accordingly, it is an object of the present invention to provide a micro-nano fluidic biochip, which is capable of uniform absorption of agents, enables the quantification of the signal of a sample, can use any pad type having a high sample absorption capacity, and makes it possible to analyze and diagnose a small amount of a sample.

In accordance with an aspect of the present invention, there is provided a micro-nano fluidic biochip comprising a second substrate disposed between a first substrate and a third substrate, in which:

the first substrate is provided on the side facing the second substrate with a reagent pad containing a reagent for analyzing a sample, an absorption pad for absorbing the sample, and a lower channel assembly for forming a microfluidic channel positioned between the absorption pad and the reagent pad,

the second substrate is provided with an upper channel assembly for forming the microfluidic channel at a position corresponding to the lower channel assembly of the first substrate and holders for holding the reagent and absorption pads on the first substrate,

the second substrate and the first substrate are joined such that the upper channel assembly and the lower channel assembly are coupled with each other, to form a microfluidic channel,

the third substrate is provided with a sample inlet that communicates with the reagent pad of the first substrate, a window disposed at a position corresponding to the microfluidic channel, and one or more vent holes that communicate with the absorption pad of the first substrate, and

the parts of the second substrate corresponding to the vent holes and the sample inlet are open;

wherein the microfluidic channel formed through joining the lower and upper channel assemblies has nano interstices formed at both sides thereof, the height of the interstices being less than that of the center of the channel.

### BRIEF DESCRIPTION OF THE DRAWINGS

The above and other objects and features of the present invention will become apparent from the following description of the invention, when taken in conjunction with the accompanying drawings, which respectively show:

FIGS. 1A and 1B: an exploded perspective view and an assembled perspective view, respectively, of a micro-nano fluidic biochip for assaying a biological sample according to an embodiment of the present invention;

FIGS. 1C and 1D: a side view and a perspective bottom view, respectively, of the biochip of FIG. 1B;

FIGS. 2A to 2H: various modifications of a microfluidic channel having nano interstices in the micro-nano fluidic biochip for assaying a biological sample according to one embodiment of the present invention;

FIG. 3A: a relation between the channel and pads disposed between a first substrate and a second substrate in the micro-nano fluidic biochip for assaying a biological sample according to the embodiment of the present invention;

FIGS. 3B to 3H: various states in which one or both of channel assemblies are subjected to surface roughness treatment, or are coated or filled with a reactive/absorptive material;

FIG. 4: a graph showing changes in strength of hepatitis signals depending on the amount of a specific component using the micro-nano fluidic biochip (FIG. 1 and FIG. 3F) according to the embodiment of the present invention, compared to results of the '862 patent; and

FIG. 5: graphs showing changes in strength of hepatitis signals depending on an analysis time using the micro-nano fluidic biochip (FIG. 1 and FIG. 3F) according to the embodiment of the present invention.

### DETAILED DESCRIPTION OF THE INVENTION

Hereinafter, a detailed description will be given of the present invention with reference to the appended drawings.

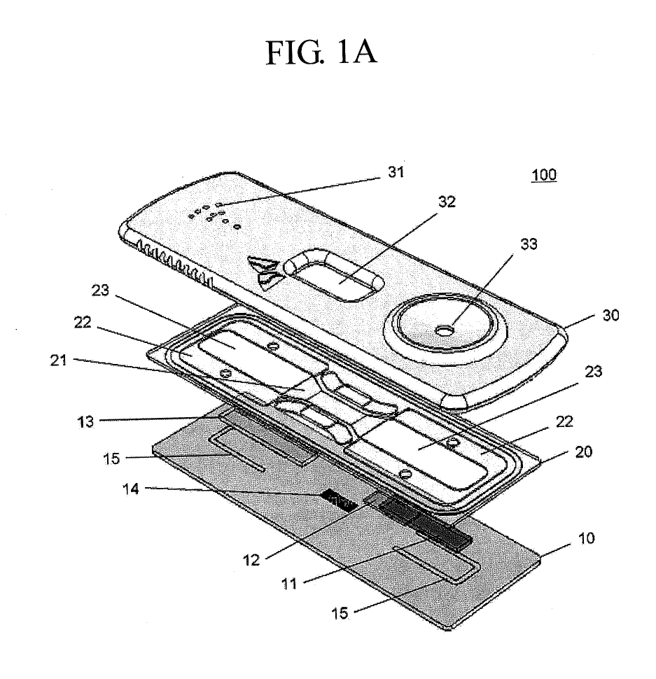

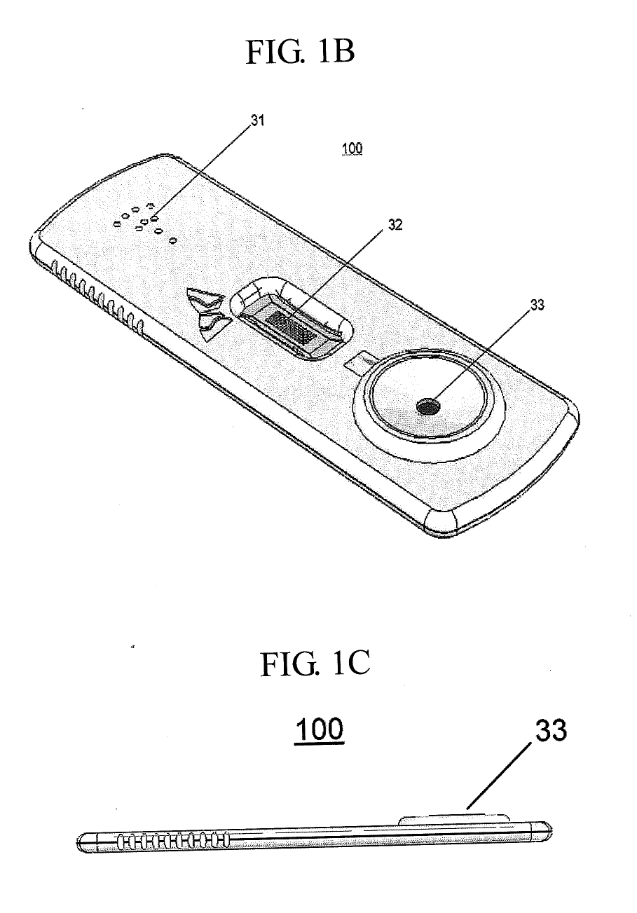

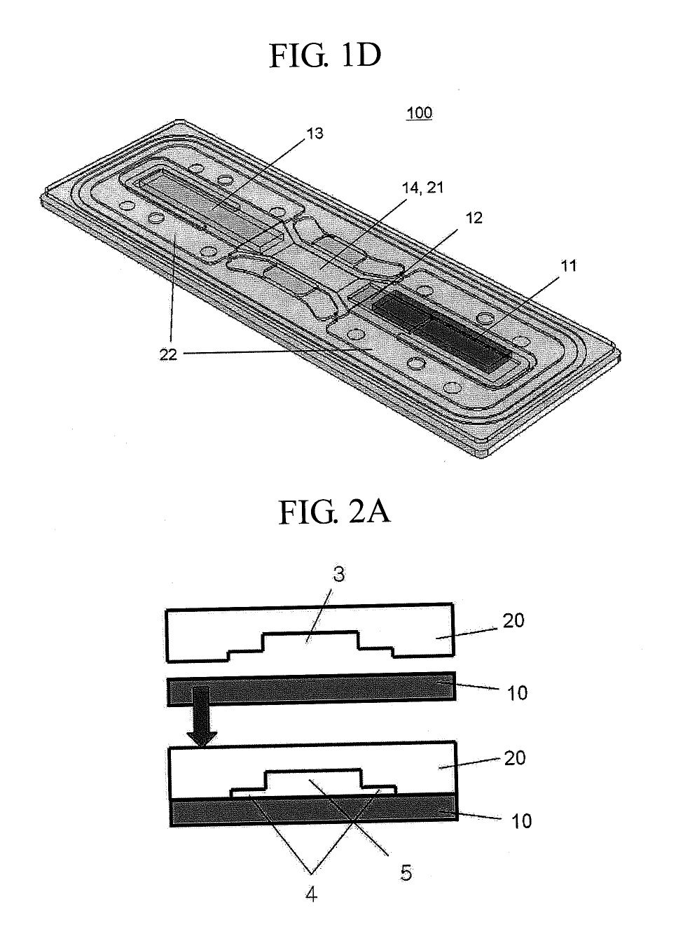

FIG. 1A is an exploded perspective view of a micro-nano fluidic biochip (100) for assaying a biological sample according to an embodiment of the present invention.

With reference to FIG. 1A, the micro-nano fluidic biochip (100) for assaying a biological sample is composed of a first substrate (10), a second substrate (20) and a third substrate (30) which are produced by injection molding of a transparent or opaque plastic. The third substrate (30) is opaque, whereas the second substrate (20) and the first substrate (10) are transparent to make it possible to conduct qualitative or quantitative analysis of a sample placed therein by measuring, e.g., the degree of color development or fluorescence emission. For example, in case when the signal is detected through the third substrate (30), the second substrate (20) should be transparent. Alternatively, in case when the second substrate (20) is opaque and the first substrate (10) is transparent, the signal is detected from the bottom side.

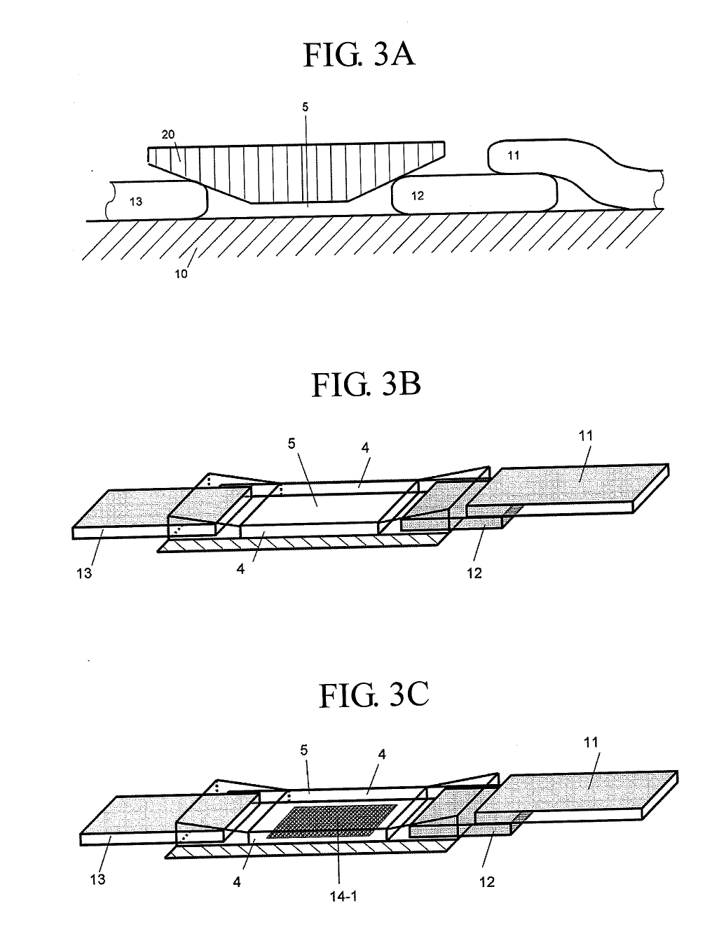

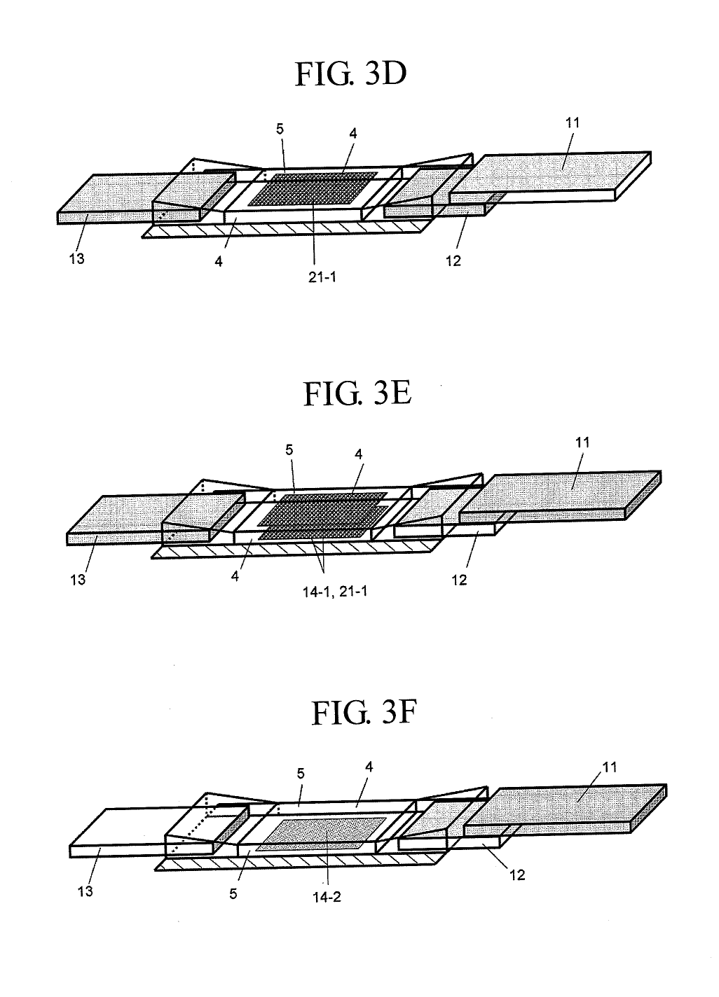

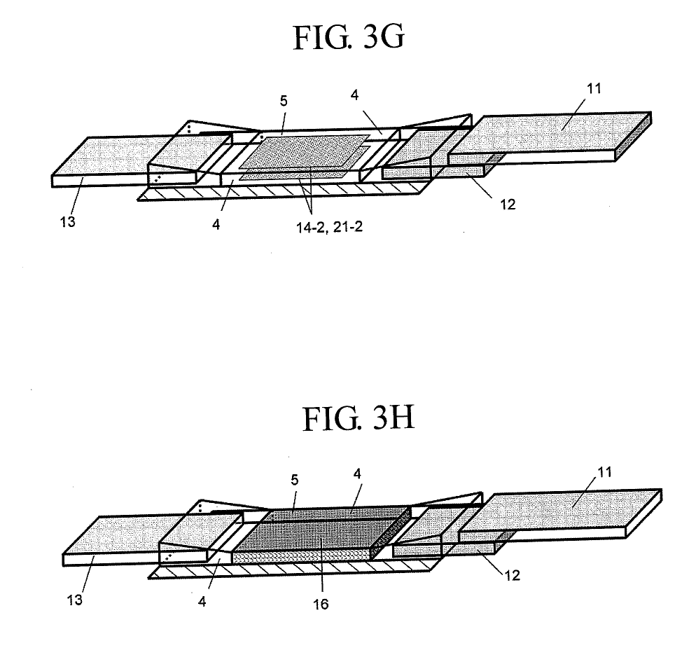

On the side of the first substrate (10) in contact with the second substrate (20), various types of pads may be formed by a diverse combination of methods as illustrated in FIGS. 3A to 3H. Referring to FIGS. 1 and 3A to 3H, examples of the pads include: an optional sample pad (11), e.g., a porous polymer (e.g., Hemasep™, CytoSep™ available from PALL) and a glass fiber pad for receiving and separating a sample transported from the sample inlet of the third substrate; a reagent pad (12) containing a color reagent such as a fluorescence reagent and a gold reagent immobilized thereon in order to detect a reactive solution; and an absorption pad (13) made of glass fiber, paper, cellulose or an absorptive polymer to control the flow rate of a fluid.

The sample pad (11) may be disposed close to the reagent pad (12), preferably in contact therewith, so that the sample first reacts with the reagent, while communicating with the sample inlet (33) of the third substrate. The absorption pad (13) is placed apart from the sample and reagent pads with a channel assembly of the first substrate disposed therebetween such that the sample reacted with the reagent can flow from the reagent pad to the absorption pad. In case when there is no need to remove undesired components from the sample through filtration, the sample pad (11) may be omitted.

The reagent pad (12) contains a color reagent, e.g., fluorescence or gold nanobeads, and when the sample flows into the reagent pad, a specific component in the sample reacts with the color reagent in the reagent pad to emit specific signals (color development or fluorescence). Such color changes may be directly observed with the naked eye (qualitative detection) or the degree of color development may be quantified using a detector. For example, in case when a fluorescence reagent is used, the intensity of light is measured quantitatively using a fluorescence detection system which is equipped with, e.g., a sensor. The amount of a specific component, present in the sample, may be measured with the signal detection system. The reacted sample is absorbed by the absorption pad (13). The sample absorbed by the absorption pad (13) is removed through vent holes (31) disposed on the third substrate (30) so that the absorptive capacity of the absorption pad (13) is restored. Both edges of the first substrate (10) are provided with a guide (15) for the pads so as to prevent the sample from leaking from the pads.

Disposed at the center of the first substrate (10), more specifically between the reagent pad (12) (or the sample pad (11)) and the absorption pad (13) is a lower channel assembly (14) which is coupled with an upper channel assembly (21) provided on the second substrate (20) to form a microfluidic channel (5).

The upper channel assembly (21) is disposed at the center of the second substrate (20), more specifically at a position corresponding to the lower channel assembly (14), and they are coupled to form a microfluidic channel (5). The second substrate (20) includes holders (22) for holding one or more pads (11, 12, 13) on the first substrate (10).

The microfluidic channel (5) has nano interstices (4) formed at both sides thereof and having a height less than that of the center of the channel. The nano interstices (4) may be pre-formed in the lower channel assembly (14) of the first substrate (10) or the upper channel assembly (21) of the second substrate (20) before joining the first and second substrates, or may be formed after joining the first and second substrates. For example, a stepped protrusion having a width of about 1 mm may be formed around the upper channel assembly (21). Then, upon joining of the second substrate (20) and the first substrate (10), only the region around the protrusion is joined, leaving a unjoined space between the second substrate and the first substrate which serves as the nano interstices (4). The nano interstices (4) thus formed may have a height ranging from 10 nm to 5 μm to ensure a stable capillary flow of the fluid, and the size of the microfluidic channel (5) is not limited but may have a dimension that enables analysis of a small amount of a sample (about 100 μl) while making the flow of the fluid efficient. For example, the dimension may have a height ranging from 5 μm to 1 mm, a length ranging from 5 mm to 40 mm and a width of less than 10 mm.

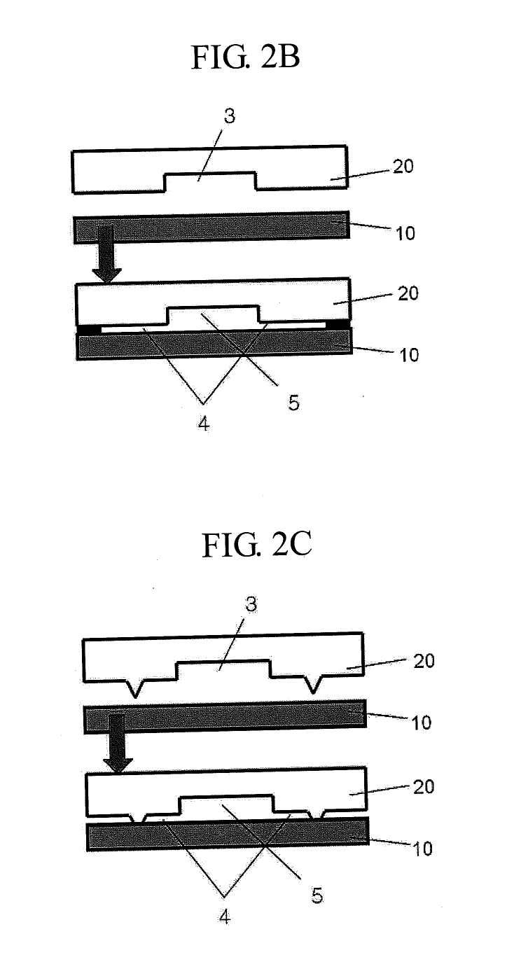

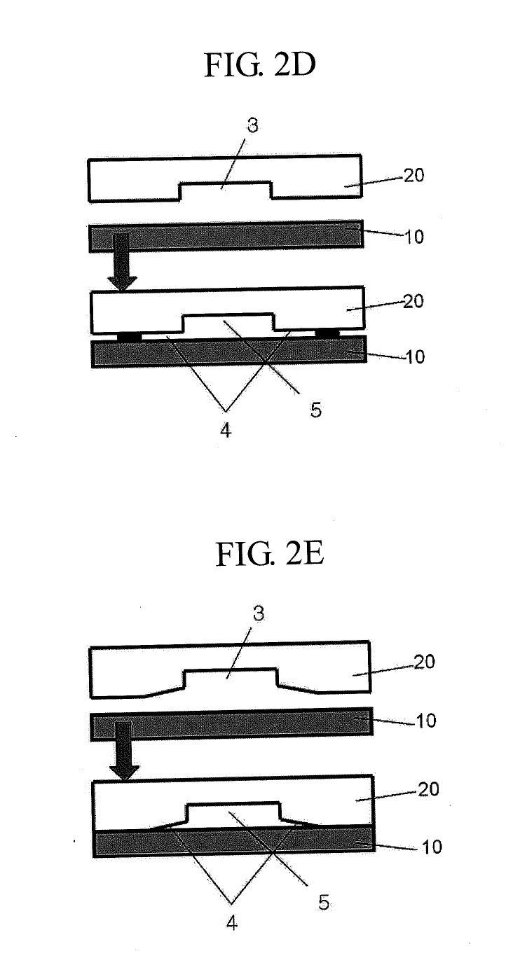

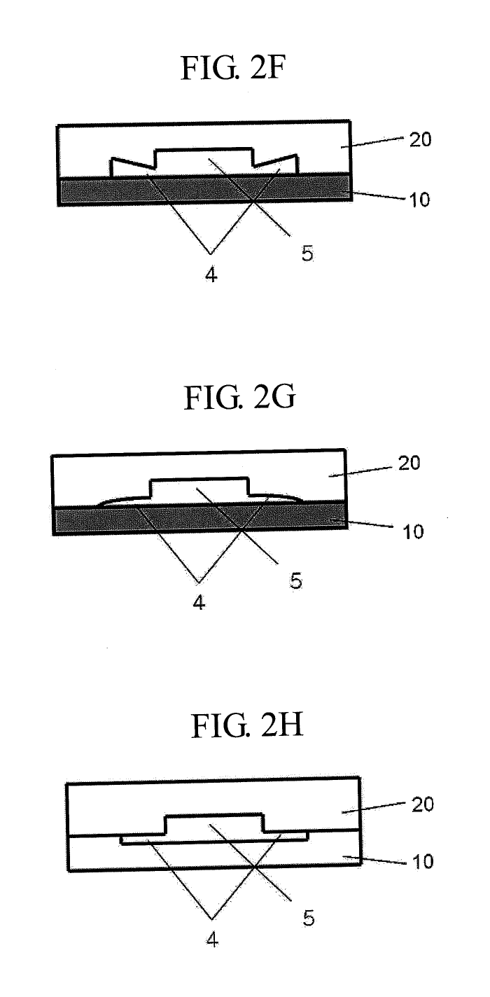

In the present invention, the first substrate (10) and the second substrate (20) are laminated vertically, compressed, and joined using a solvent joining process, an ultrasonic joining process, an adhesive joining process, a tape joining process, a heat joining process, a pressure joining process, or a laser joining process. As a result, the microfluidic channel having the nano interstices formed at both sides thereof, which is connected to the pads (11, 12 and 13) and has a height less than that of the center thereof is formed. Various modifications thereof are illustrated (FIGS. 2A to 2H).

In order to enhance the quantitative analysis capability of a sample and/or the reactive area, one or both of the upper channel assembly (21) and the lower channel assembly (14) may be subjected to oxygen plasma treatment to confer thereon an average surface roughness of less than 10 μm (14-2, 21-2). Also, pillar structures having various cross-sectional shapes or nano-groove patterns may be formed to construct fine structures (14-1, 21-1) having an increased surface area. Also, one or both of the upper channel assembly (21) and the lower channel assembly (14) may be coated with a metallic thin film (e.g., gold, silver and platinum) or an absorptive thin film (e.g., cellulose). Also, a reactive or absorptive material (16), e.g., cellulose and glass fiber, may be loaded between the upper and lower channel assemblies of the microfluidic channel to enhance the reactivity. Various states described above for the upper and lower channel assemblies are illustrated in FIGS. 3C to 3H. In case the pillar structures (14-1) having dots are formed on the lower channel assembly (14), the surface area of the first substrate (10) increases, and the reaction of individual dots is detected using a general scanner, thereby maximizing the quantification accuracy.

The first and second substrates housing the microfluidic channel may be made of any material typically used in the art, e.g., silicon, glass, pyrex, PDMS (polydimethylsiloxane), plastic, etc.

The third substrate (30) is disposed on the second substrate (20) of the joined laminate of the first and second substrates (10 and 20) to complete the micro-nano fluidic biochip (100) for assaying a biological sample according to the present invention (FIG. 1B).

The third substrate (30) is provided with one or more vent holes (31) for discharging air from the device to control the flow of the fluid on the absorption pad (13) of the first substrate (10) (namely, to enhance the flow and loading of a sample into the channel assemblies (14, 21)), a sample inlet (33) for injecting a sample to be assayed, and a window (32) disposed between the vent holes and the sample inlet. The vent holes and the sample inlet are separated from each other by a predetermined distance so that the vent holes and the sample inlet are respectively disposed to communicate with the absorption pad (13) and the reagent pad (12) (or the sample pad (11)) of the first substrate (10). To allow the sample to flow from the inlet to the sample pad, parts (23) are provided on the second substrate (20) at positions corresponding to the vent holes (31) and the sample inlet (33). The window (32) is disposed at a position corresponding to the microfluidic channel.

FIG. 1B is an assembled perspective view of the biochip (100) for assaying a biological sample according to the present invention; FIG. 1C, a side view of the biochip of FIG. 1B (having a total thickness of about 3 mm); and FIG. 1D, a perspective bottom view of the biochip of FIG. 1B.

The sample used in the present invention may be any inorganic or organic sample, and preferably includes a biological sample such as blood, body fluid, urine and saliva. Accordingly, the inventive micro-nano fluidic biochip which can employ a protocol for manufacturing conventional diagnostic kits can be applied to various fields for analysis and/or diagnosis of a sample, e.g., biosensors, DNA analysis chips, protein analysis chips, lab-on-a-chips, and cell counting devices.

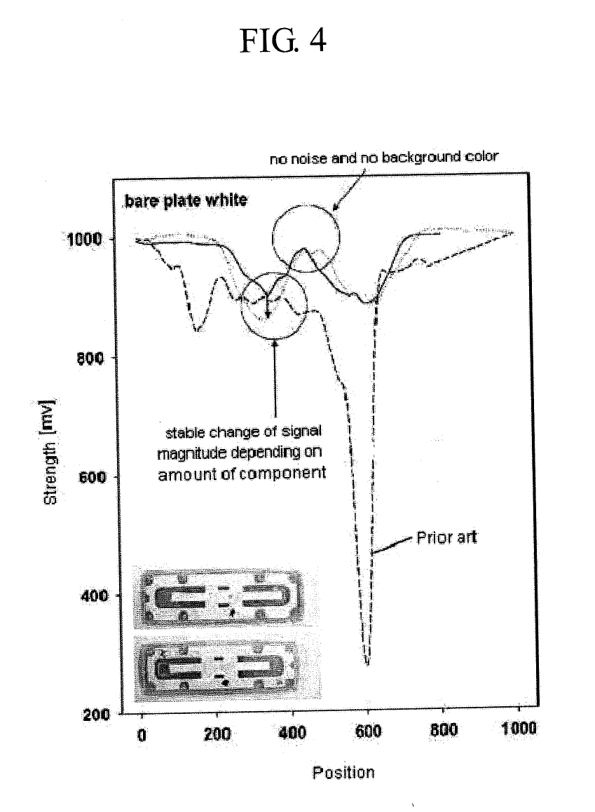

The changes in strength of hepatitis signals depending on the amount of a specific component using the inventive micro-nano fluidic biochip (100) (FIGS. 1 and 3F (using a gold reagent pad)) are shown in FIG. 4, compared to the results of the '862 patent.

In FIG. 4, the straight line and the thin dotted line (containing a double amount of a hepatitis-related component) show the signals of the hepatitis-related component in the blood sample detected using the inventive biochip, and the thick dotted line, those detected using the conventional biochip (the '862 patent) comprising a membrane.

With reference to FIG. 4, in case when a specific component is present in an approximately double amount in the sample, it is found in the inventive biochip that the strength of signals increases by approximately double without noise. However, in the conventional biochip, it is confirmed that extreme noise occurs between the first and second signals due to attachment of the gold reagent.

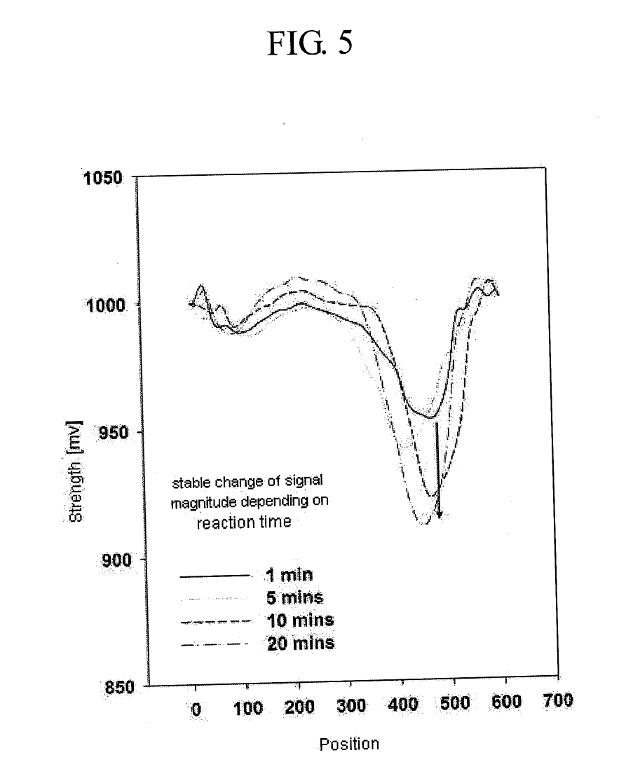

The changes in strength of hepatitis signals depending on the analysis time using the inventive micro-nano fluidic biochip (100) (FIGS. 1 and 3F) are shown in FIG. 5, which suggests that increase in the analysis time results in increase in the strength of hepatitis signals with no background signals and no noise.

As described above, the micro-nano fluidic biochip of the present invention is capable of uniform absorption of agents, enables the quantification of the signal of a sample, can use any pad type having a high sample absorption capacity, and makes it possible to analyze and diagnose a small amount of a sample. Accordingly, the inventive biochip can be advantageously used as a biosensor, a DNA analysis chip, a protein analysis chip, a lab-on-a-chip, and a cell counting device.

While the invention has been described with respect to the above specific embodiments, it should be recognized that various modifications and changes may be made to the invention by those skilled in the art which also fall within the scope of the invention as defined by the appended claims.

What is claimed is:

1. A micro-nano fluidic biochip comprising a second substrate disposed between a first substrate and a third substrate, in which:
the first substrate is provided on the side facing the second substrate with a reagent pad containing a reagent for analyzing a sample, an absorption pad for absorbing the sample, and a lower channel assembly for forming a microfluidic channel positioned between the absorption pad and the reagent pad,
the second substrate is provided with an upper channel assembly for forming the microfluidic channel at a position corresponding to the lower channel assembly of the first substrate and holders for holding the reagent and absorption pads on the first substrate,
the second substrate and the first substrate are joined such that the upper channel assembly and the lower channel assembly are coupled with each other, to form a microfluidic channel,
the third substrate is provided with a sample inlet that communicates with the reagent pad of the first substrate, a window disposed at a position corresponding to the microfluidic channel, and one or more vent holes that communicate with the absorption pad of the first substrate, and
the parts of the second substrate corresponding to the vent holes and the sample inlet are open;
wherein the microfluidic channel formed through joining the lower and upper channel assemblies has nano interstices formed at both sides thereof, the height of the interstices being less than that of the center of the channel.

2. The micro-nano fluidic biochip of claim 1, wherein the first substrate further comprises a sample pad for receiving and separating a sample transported from the sample inlet of the third substrate, which is disposed close to the reagent pad.

3. The micro-nano fluidic biochip of claim 1, wherein the center of the microfluidic channel has a height ranging from 5 μm to 1 mm, and each of the nano interstices has a height ranging from 10 nm to 5 μm.

4. The micro-nano fluidic biochip of claim 1, wherein the sample pad is a porous polymer pad or a glass fiber pad.

5. The micro-nano fluidic biochip of claim 1, wherein the reagent pad contains a fluorescence reagent or a gold reagent immobilized thereon.

6. The micro-nano fluidic biochip of claim 1, wherein the absorption pad is an absorptive polymer pad or a glass fiber pad.

7. The micro-nano fluidic biochip of claim 1, wherein the biochip is selected from the group consisting of a biosensor, a DNA analysis chip, a protein analysis chip, a cell counting device, and a lap-on-a chip.

8. The micro-nano fluidic biochip of claim 1, wherein one or both of the upper channel assembly and the lower channel assembly are formed with pillar structures having various cross-sectional shapes or nano-groove patterns.

9. The micro-nano fluidic biochip of claim 1, wherein one or both of the upper channel assembly and the lower channel assembly are subjected to plasma treatment to confer thereon an average surface roughness of less than 10 μm.

10. The micro-nano fluidic biochip of claim 1, wherein one or both of the upper channel assembly and the lower channel assembly are coated with a metallic thin film.

11. The micro-nano fluidic biochip of claim 1, wherein one or both of the upper channel assembly and the lower channel assembly are coated with an absorptive thin film.

12. The micro-nano fluidic biochip of claim 1, wherein a reactive or absorptive material is loaded between the upper channel assembly and the lower channel assembly of the microfluidic channel.

13. The micro-nano fluidic biochip of claim 1, wherein the nano interstices are pre-formed in the upper channel assembly of the second substrate or in the lower channel assembly of the first substrate before joining the second substrate and the first substrate, or are formed after joining the second substrate and the first substrate.

14. The micro-nano fluidic biochip of claim 1, wherein the joining process is selected from the group consisting of a solvent joining process, an ultrasonic joining process, an adhesive joining process, a tape joining process, a heat joining process, a pressure joining process, and a laser joining process.
